(19) United States
(12) Patent Application Publication
(10) Pub. No.: US 2007/0264313 A1
(43) Pub. Date: Nov. 15, 2007

(54) Methods and compositions relating to animal dietary acids

(76) Inventors: Robert E. Musser, Good Thunder, MN (US); Giancarlo Alloa Casale, Monaco (MC)

Correspondence Address: DORSEY & WHITNEY LLP;INTELLECTUAL PROPERTY DEPARTMENT, SUITE 1500, 50 SOUTH SIXTH STREET, MINNEAPOLIS, MN, 55402-1498, US

(21) Appl. No.: 11/436,391

(22) Filed: May 18, 2006

Related U.S. Application Data
(60) Provisional application No. 60/799,888, filed on May 12, 2006.

(51) Int. Cl. A61K 47/00; A61K 33/14; A61K 31/19

(52) U.S. Cl. 424/439; 514/574; 424/679; 424/442

(57) ABSTRACT

The present invention relates to an animal dietary composition including a dietary acid and further to methods of feeding an animal that include providing a dietary acid as part of the animal's diet, resulting in enhanced feed efficiency and weight gain. According to one embodiment, the present invention further relates to limiting the animal's diet.

### CROSS-REFERENCE TO RELATED APPLICATION(S)

This application claims the benefit of and priority to U.S. provisional patent application Ser. No. ______, filed May 12, 2006, entitled METHODS AND COMPOSITIONS RELATING TO ANIMAL DIETARY ACIDS, inventor is Robert E. Musser, which is incorporated by reference herein in its entirety.

### FIELD OF THE INVENTION

The present invention relates to methods for using and compositions of a dietary acid and further to enhancing the feed efficiency and weight gain of an animal by providing a dietary acid to the animal. In addition, the invention further relates in certain embodiments to restricting the animal's diet.

### BACKGROUND OF THE INVENTION

A primary focus of animal production is improved feed efficiency, body composition, and reproductive capacity. Feed efficiency is the amount of gain exhibited by an animal in comparison to the amount of feed consumed.

In swine, one focus of feed efficiency is the late finishing period, during which swine exhibit poor conversion of feed to weight gain. Because of this poor feed efficiency, the cost to add weight is higher in this stage of growth than earlier in the pig's life and further is higher in comparison to the feed efficiency of other protein sources, such as fish and chicken. In addition, improvements in feed efficiency can be economically beneficial for sows during the lactation period, resulting in improved sow and litter weights.

Thus, there is a need in the art for methods and compositions for improving animal feed efficiency.

### BRIEF SUMMARY OF THE INVENTION

The present invention, in one embodiment, is an animal dietary supplement that includes a dietary acid and a diet restriction component. In one aspect of the invention, the dietary acid is configured such that a portion of the dietary acid is active in the large intestine of the animal. In another embodiment, the supplement also includes a protection component.

The present invention, in another embodiment, is an animal dietary supplement that includes a dietary acid, a diet restriction component and a protection component. The diet restriction component is potassium chloride, bitter feed flavors, aromatics, salt, fat, distiller's grains, meat meal, or fish oil.

In another embodiment, the present invention is a method of feeding an animal. The method includes providing a dietary acid to an animal as part of a restricted diet.

The present invention, according to another embodiment, is a method of improving average daily gain in swine. The method includes providing a dietary acid to a finishing swine as part of a restricted diet.

In accordance with another embodiment, the present invention is a method of improving feed efficiency in swine. The method includes implementing a limited diet for a finishing swine and supplementing the limited diet with a dietary acid.

According to a further embodiment, the present invention is a method of improving feed efficiency in swine. The method includes providing a dietary acid to a finishing swine as part of a restricted diet. In one embodiment, the dietary acid is encapsulated. In another embodiment, the dietary acid is not fully released until it reaches a large intestine of the swine.

While multiple embodiments are disclosed, still other embodiments of the present invention will become apparent to those skilled in the art from the following detailed description, which shows and describes illustrative embodiments of the invention. As will be realized, the invention is capable of modifications in various obvious aspects, all without departing from the spirit and scope of the present invention. Accordingly, the drawings and detailed description are to be regarded as illustrative in nature and not restrictive.

### BRIEF DESCRIPTION OF THE DRAWINGS

FIG. 1 is a schematic side view of the digestive system of a pig.

### DETAILED DESCRIPTION

The methods and compositions of the present invention relate to enhancing feed efficiency in animals, including non-ruminants. In one embodiment, the animals are finishing swine. In another embodiment, the animals are lactating sows. More specifically, the present invention relates to dietary acid compositions and methods of feeding those compositions to animals. According to one embodiment, the compositions of the present invention include a feed supplement containing a dietary acid and, in some embodiments, a diet restriction component. In another embodiment, the methods of the present invention include restricting or limiting nutritional components of the diet in combination with providing a dietary acid composition which, according to certain embodiments, is configured such that at least a portion of the composition is active in the large intestine.

According to one embodiment, the composition of the present invention includes a dietary acid. For purposes of this application, “dietary acid” (also referred to herein as “acidifier”) is intended to include any organic and/or inorganic acid that can be consumed by an animal. In one aspect of the invention, the dietary acid is fumaric acid. Alternatively, the dietary acid is any of, but not limited to, fumaric acid, malic acid, citric acid, phosphoric acid, lactic acid, formic acid, acetic acid, propionic acid, butyric acid, sorbic acid, tartaric acid, benzoic acid, or 2-hydroxy-4-(methylthio)butanoic acid, or any combination thereof. The dietary acid, according to one embodiment, is a combination of dietary acids in which fumaric acid comprises an amount ranging from about 1% to about 100% of the dietary acid. Alternatively, the fumaric acid comprises an amount ranging from about 15% to about 75% of the dietary acid. In a further alternative, the fumaric acid ranges from about 30% to about 50% of the dietary acid.

In a further alternative embodiment, the combination of dietary acids includes malic acid. In one aspect of the invention, the malic acid is present in an amount ranging from about 0% to about 99% of the dietary acid. Alternatively, malic acid is present in an amount ranging from about 5% to about 60% of the dietary acid. In a further alternative, malic acid is present an amount ranging from about 10% to about 30% of the dietary acid.

In another alternative embodiment, the combination of dietary acids includes citric acid. According to one embodiment, the citric acid is present in an amount ranging from about 0% to about 99% of the dietary acid. Alternatively, citric acid is present in an amount ranging from about 5% to about 60% of the dietary acid. In a further alternative, citric acid is present in an amount ranging from about 10% to about 30% of the dietary acid.

According to a further alternative embodiment, the combination of dietary acids includes phosphoric acid. In one aspect, the phosphoric acid is present in an amount ranging from about 0% to about 99% of the dietary acid. Alternatively, phosphoric acid is present in an amount ranging from about 5% to about 60% of the dietary acid. In a further alternative, phosphoric acid is present in an amount ranging from about 10% to about 30% of the dietary acid. In one embodiment, the phosphoric acid is orthophosphoric acid.

In accordance with yet another alternative embodiment, the combination of dietary acids includes lactic acid. The lactic acid is present in an amount ranging from about 0% to about 50% of the dietary acid. Alternatively, the lactic acid is present in an amount ranging from about 0.01% to about 20%. In a further alternative, the lactic acid is present in an amount ranging from about 0.1% to about 10

According to one alternative embodiment of the present invention, the dietary acid is a combination of dietary acids comprising the following: about 40.6% fumaric acid, about 20.3% malic acid, about 20.3% citric acid, about 20.3% phosphoric acid, and about 0.2% lactic acid.

In one aspect of the invention, the composition is a feed additive or supplement providing for a dietary acid or combination of dietary acids added to feed in an amount ranging from about 0.25 lbs to about 20 lbs per ton of feed. Alternatively, the dietary acid is added to feed in an amount ranging from about 0.5 lbs to about 15 lbs per ton of feed. In a further alternative, the acid is added to feed in an amount ranging from about 1 lb to about 10 lbs per ton of feed.

According to one embodiment, the composition is a feed additive or supplement that includes both a dietary acid (or acids) and at least one additional component. In this embodiment, the dietary acid or acids are present in the composition in an amount ranging from about 1% to about 99% by weight of the composition. Alternatively, the acid or acids are present in an amount ranging from about 30% to about 70% by weight. In a further alternative, the acid or acids are present in an amount ranging from about 40% to about 60% by weight.

In one aspect of the invention in which the composition comprises a dietary acid and at least one additional component, the composition includes a diet restriction component. For purposes of the present application, a “diet restriction component” (also referred to herein as a “diet limitation component,” “diet suppressant,” “intake suppressant,” “appetite suppressant,” and equivalents) is any edible component that can be added to the composition of the present invention for purposes of reducing an animal's feed intake. Reduced feed intake can be measured by several methods known in the art, including reduction from an ad libitum consumption amount. In one aspect of the invention, the intake suppressant is a beta-adrenergic agonist. For example, in one embodiment the intake suppressant is ractopamine hydrochloride. According to one embodiment, the ractopamine hydrochloride is present in an amount ranging from about 2 to about 9 grams/ton (2.5 to 10 grams/metric tonne) of complete feed. In one embodiment, the ractopamine hydrochloride is provided in the form of PayLean®, which is available from Elanco, located in Greenfield, Ind.

For purposes of the remainder of the application, all units of measure shall be set forth in standard English/American units. It is understood that these units can be easily converted into metric units of measure.

Alternatively, the intake suppressant is potassium chloride. In one aspect, the potassium chloride is present in an amount ranging from about 1 to about 10 lbs/ton of complete feed. In a further alternative embodiment, the intake suppressant can be bitter feed flavors such as, for example, saccharine, in an amount that is greater than about 0.01% by weight of the complete feed. Alternatively, the intake suppressant is sodium chloride in an amount that is either (1) about 0.25% by weight or less of the complete feed or (2) about 5% by weight or more of the complete feed. The intake suppressant in another embodiment is fat, including, but not limited to, choice white grease, poultry fat, tallow, vegetable oil, or mineral oil. In yet another embodiment, the intake suppressant can be distiller's grains, either (1) in an amount that is at least about 5% of the complete feed or (2) having a lysine digestibility of about 55% or less. Alternatively, the suppressant is meat meal in an amount ranging from about 5% to 15% by weight of the complete feed. In a further alternative, the suppressant is fish oil in an amount ranging from about 5 to about 100 lbs/ton of complete feed. According to one aspect of the invention, the suppressant is a high fiber in an amount that is about 3% or more of the complete feed. Alternatively, the diet restriction component can be any combination of the suppressants discussed herein. In a further alternative, any of the above diet restriction components or any combination thereof can be provided in combination with any of the diet restriction methods described herein. In yet another alternative, the diet restriction component can be any known component or composition known to reduce feed intake in an animal.

The composition, in accordance with another embodiment, can include additional ingredients. In one aspect of the invention, the ingredients are any known ingredients helpful as a carrier or other substance that helps maintain the form or viability of the composition. Alternatively, one or more additional ingredients may enhance the benefits of the composition. For example, according to one embodiment, the composition includes a carrier component. In one embodiment, the carrier component is calcium pantothenic acid. Other components that may provide textural, viability, or enhancement benefits include rice hulls, calcium carbonate, mineral oil, oligosaccharides (such as inulin, which is commercially available in a product called Ultra-fos®, available from Encore Technologies, LLC, located in Minneapolis, Minn.), L-carnitine and chromium picolinate (such as Carnichrome® 50, available from Lonza, located in Fairlawn, N.J.), niacinamide, and/or biotin premix.

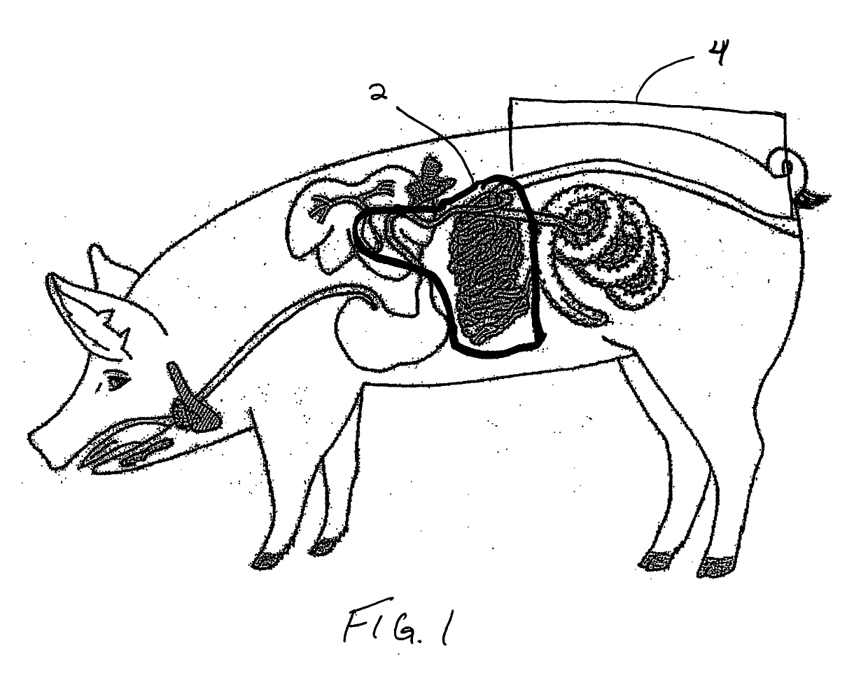

The composition, according to one embodiment, is configured such that upon consumption, a portion of the composition is available and active in the large intestine (also referred to herein as the “hind gut”). That is, the composition is configured such that it is not fully released in the small intestine or is “functionally delayed.” For purposes of clarification, FIG. 1 provides a side view of the digestive system of a pig. The small intestine 2 is comprised of the duodenum, the jejunum, and the ileum. The large intestine or hind gut 4 includes the cecum, the colon, and the rectum. For purposes of this application, a “functionally delayed” or “delayed action” composition is any composition of the present invention that is configured such that it is not fully digested before or in the small intestine 2 and thus is active or effective in both the small 2 and large 4 intestines.

Without being limited by theory, it is believed that the dietary acid acts in the small intestine 2 to enhance nutrient digestion and further acts in the large intestine 4 to provide some control of bacterial pathogens. It is also believed that the benefits of ensuring the presence of active dietary acid in the large intestine relate to the avoidance of the buffering effects in the small intestine of non-ruminants. That is, acids entering the digestive system of a non-ruminant will generally be buffered in the stomach by other dietary ingredients or will have limited effect because of decreased gastric acid secretions. Further, acidic chyme coming from the stomach into the proximal portion of the duodenum in the small intestine is buffered by bile secretions entering the duodenum from the gall bladder. It is believed that one benefit of the present invention relates to the delayed activation of the composition such that active dietary acid passes through the small intestine without being completely buffered and thus results in the presence of the dietary acid in the large intestine in an active or effective amount, thereby enhancing the effects of the dietary acid in the digestive system.

In one embodiment, a protection component is added to the composition to prevent complete release in the small intestine. The protection component can be associated with the dietary acid or with the composition as a whole. In one aspect of the invention, the protection component is an encapsulation layer added to the composition and comprising a lipid, fat, or oil. For example, according to one embodiment, the encapsulation layer comprises food grade hydrogenated vegetable oils that are applied to the composition of the present invention. Alternatively, the encapsulation layer can include one or more of the following in any combination: wax, silicone, enteric polymers, cellulose derivatives, vinyl derivatives, acrylic derivatives, gelatin, or starch.

In one embodiment, the layer components are applied by a spray freezing process. Alternatively, they are applied by a spray cooling process. In a further alternative, the encapsulation components can be applied by any known process for encapsulating an edible composition.

According to an alternative embodiment, the protection component incorporated into the composition is any component or composition that will provide a delayed release of the composition.

According to one embodiment, the composition of the present invention is a supplement that can be added to, and is sold separately from, a complete feed. For purposes of this application, “complete feed” is a term that is known in the art and includes any feed provided to an animal or animals that comprises all of the essential nutrients for the animal. According to one embodiment, the complete feed is the only food provided to the animal(s) (other than the supplement of the present invention and any other supplements that are known to be added to a complete feed).

Alternatively, the composition is included as a component of a complete feed product. In a further alternative, the composition takes any known form, including solid or liquid form, and can be used in any way known in the art. For example, according to one embodiment, the composition can be, but is not limited to, a component of a base mix, a premix, a concentrate, a vitamin trace mineral mix (“VTM”), or in a solution delivered through the animal's water supply.

In one aspect of the invention, the composition includes a commercially available encapsulated product containing dietary acids called Aciprol®), which is available from SODA Feed Ingredients, located in Monaco. According to one embodiment, the composition further includes additional components as described herein.

The method of the present invention, in accordance with one embodiment, relates to feeding a dietary acid composition such as any of the compositions described herein to an animal to enhance the animal's feed efficiency. That is, the method relates to providing a dietary acid composition to an animal such that the animal may gain weight more quickly, or gains the same weight while consuming less feed, or gains more weight while consuming the same amount of feed, than if the dietary acid composition were not provided. These feed efficiency benefits can be exhibited by such calculations as the animal's average daily gain (“ADG”), feed to gain ratio (“feed:gain”), gain to feed ratio (“gain:feed”), and average daily feed intake (“ADFI”).

It is understood that the methods of the present invention can be practiced with respect to any kind of animal, including non-ruminants. It is further understood that the methods are practiced according to one embodiment, with respect to swine. Alternatively, the methods are practiced with respect to poultry. In a further alternative, the methods are practiced with any known animal.

Without being limited by theory, it is believed that the addition of the acidifier as a dietary supplement benefits feed efficiency and average daily weight gain through its impact on certain digestive enzymes and organisms present in the digestive system. More specifically, the presence of the acidifier may stimulate or activate the enzymes, thereby improving the efficiency of digestion and use of the consumed nutrients. Further, the presence of the acidifier may inhibit the ability of certain organisms present in the digestive tract such as E. coli to consume or scavenge the available nutrients. This inhibition may result from certain organisms' aversion to acidic environments.

According to another embodiment, the method of the present invention relates to feeding a dietary acid composition such as any of the compositions described herein to an animal to enhance the animal's feed efficiency while lactating. That is, the method relates to providing a dietary acid composition to a lactating female animal such that the animal may maintain better body condition (lose less weight, maintain weight, or gain weight) during lactation. Also, providing the composition to the female can result in at least two benefits to the female's offsping in comparison to providing a diet without the composition: (1) increased weight at time of weaning, and (2) lower mortality. For the female, these feed efficiency benefits can be exhibited by such calculations as change in body weight or composition (body protein or fat), percent animals return to estrus, days to estrus, and percent animals rebred. For the offspring, these feed efficiency benefits can be exhibited by such calculations as number of pigs weaned, percent mortality, litter weight gain, and weaning weight.

In one aspect of the invention, the method includes feeding to an animal a dietary acid composition according to any embodiment of the composition provided herein. In one aspect of the invention, the method includes feeding such a dietary acid composition to finishing swine. For purposes of this application, “finishing swine” (also referred to herein as “growing swine,” “growing/finishing swine,” “growers,” “finishers,” and “grower/finishers”) is intended to include any pig that is older than about 70 days or that has a weight of at least about 50 pounds. The composition is added as a supplement to the feed of the finishing swine. According to one embodiment, the composition is included as a supplement in feed that is continuously available to the swine. Alternatively, the composition is included as a supplement in substantially regularly scheduled rations to the swine.

In one embodiment of the present invention, the method of the present invention also includes adding PayLean® to the animal's diet, as an additional supplement (in addition to the composition of the present invention) to the complete feed of the animal. Alternatively, PayLean® is added to the composition of the present invention. PayLean®), as discussed above, is a source of ractopamine hydrochloride. The combination of the dietary acid composition of the present invention and PayLean® exhibits synergistic beneficial effects relating to feed efficiency and average daily gain. Without being limited by theory, it is believed that the ractopamine hydrochloride present in PayLean® provides diet limitation benefits that may create the synergy. Alternatively, the method can include adding any beta-adrenergic agonist to the animal's diet.

According to another embodiment, the method further includes limiting or restricting the diet of the animal. That is, the feed or nutrients provided to the animal are rationed to the animal in a fashion such that the animal consumes less than the animal would typically consume. In one aspect of the invention, the entire diet of the animals is restricted by some amount. That is, the amount of feed made available to the animal is reduced. In one embodiment, the daily diet of the animal is reduced by an amount ranging from about 1% to about 50% by weight of the animal's typical daily consumption. Alternatively, the diet is reduced by an amount ranging from about 5% to about 30% by weight of the animal's typical daily consumption.

According to one embodiment, the diet restriction can be achieved by limiting the availability of feed, as discussed above. In one aspect relating to limiting the availability, the animal is not allowed to consume feed ad libitum. Alternatively, the diet restriction can be achieved by other methods. In one aspect of the invention, the diet is restricted or limited by increasing the number of animals in the pen or confinement space. By increasing the number of animals in the pen, the feed is less accessible to each animal. In embodiments in which feed is continuously available to the animals, another method is to limit the feeder space available to each of the animals. Alternatively, the amount of water available to the animals can be restricted. This water restriction reduces the animals' feed intake.

In a further alternative, the method of the present invention relates to restricting or limiting or reducing a specific portion of the animal's diet. For example, according to one embodiment, the protein portion of the animal's diet is restricted. For purposes of the present invention, the “protein portion of an animal's diet” means the nitrogen containing portion of the diet made up of essential and nonessential amino acids. Alternatively, the energy portion of the animal's diet is restricted. For purposes of the present invention, the “energy portion of the animal's diet” means any portion of the diet that provides energy used to keep the animal in energy equilibrium, maintain normal body functions, do work, or for synthesis of tissue, milk, fetus, eggs, hair, etc., or for the production of heat. In a further alternative, both the protein and energy portions are restricted.

Alternatively, an appetite suppressant is included with the dietary acid as described above. According to one embodiment, the suppressant is premixed with the acid. Alternatively, the suppressant is added along with the dietary acid(s) as supplements to the animal's feed. In a further alternative, the suppressant can be provided by any known method and in any known form.

In one aspect of the invention, the beneficial effects of the dietary acid, especially with respect to feed efficiency, are enhanced when provided in combination with a limited or restricted diet.

Although the present invention has been described with reference to preferred embodiments, persons skilled in the art will recognize that changes may be made in form and detail without departing from the spirit and scope of the invention.

The invention will now be described further by way of the following examples.

### EXAMPLE 1

In this example, the efficacy of a dietary acid composition in combination with a ractopamine hydrochloride product was examined.

Methods and Materials

A total of 180 pigs were used in this experiment, with the initial weight of the pigs at 198 lbs. Treatments consisted of one of 4 feeding programs as set forth in Table 1. In addition to the Control Sample, Sample 1 contained a dietary acid supplement of the present invention. In this example, the dietary acid supplement was commercially-available Opti-Pak Efficiency™, available from Hubbard Feeds in Mankato, Minn. Further, Sample 2 contained ractopamine hydrochloride in the form of commercially-available PayLean®), and Sample 3 contained a combination of both the dietary acid and the ractopamine hydrochloride.

TABLE 1

| | | | | Sample 3 |
|---|---|---|---|---|
| | | Sample 1 | Sample 2 | (Dietary Acid & |
| | Control | (Dietary | (Ractopamine | Ractopamine |
| Ingredients (in lbs.) | Sample | Acid) | Hydrochloride) | Hydrochloride) |
| Corn | 1578.8 | 1573.8 | 1456.5 | 1451.5 |
| Limestone | 17.9 | 17.9 | 18.0 | 18.0 |
| Soybean meal | 312 | 312 | 436 | 436 |
| Lysine HCL | 1.0 | 1.0 | 1.0 | 1.0 |
| Salt | 7.0 | 7.0 | 7.0 | 7.0 |
| Choice White Grease | 60 | 60 | 60 | 60 |
| Monocal Phosphate, 21% | 18.3 | 18.3 | 16 | 16 |
| Hubbard VTM | 5.0 | 5.0 | 5.0 | 5.0 |
| PayLean ® 9 gram* | 0 | 0 | 0.5 | 0.5 |
| Opti-Pak Efficiency ™ | 0 | 5.0 | 0 | 5.0 |

*Adds 9 grams per ton of complete feed when added at a rate of 1 pound per ton.

The feed program was divided into two phases as set forth in Table 2. In Phase 1, each of the four groups of pigs were fed either the Control Sample or Sample 1. During Phase 2, each group was fed a different sample as described in Table 1.

TABLE 2

| | Control | | | |
|---|---|---|---|---|
| | Group | | | |
| | Pigs fed | Group 1 | Group 2 | Group 3 |
| | Control | Pigs fed | Pigs fed | Pigs fed |
| | Sample | Sample 1 | Sample 2 | Sample 3 |
| Number of | 43 | 43 | 44 | 44 |
| pigs | | | | |
| Diet during | Control | Sample 1 | Control | Sample 1 |
| Phase 1 | Sample | | Sample | |
| (days 0-14) | | | | |
| Diet during | Control | Sample 1 | Sample 2 | Sample 3 |
| Phase 2 | Sample | | | |
| (days 15-35) | | | | |

Results

In each case, various calculations (performance data) were made for each group at the end of Phase 2, such as average daily gain, feed:gain ratios, and average daily feed intake, as shown below in Table 3. Group 1 showed significant improvements over the Control Group in feed to gain ratio, and average daily feed intake. In addition, Group 3 showed significant improvements over the other three groups in average daily feed intake, and a significant improvement over the controls with respect to feed to gain ratio.

TABLE 3

| | Control | | | |
|---|---|---|---|---|
| | Group | Group 1 | Group 2 | Group 3 |
| ADG | 2.02 | 2.03 | 2.03 | 2.20 |
| F/G | 3.54 | 3.33 | 3.35 | 3.12 |
| ADFI | 7.08 | 6.71 | 6.76 | 6.82 |

The following carcass information was also examined as set forth in Table 4: percentage lean, loin depth, and backfat depth. Group 3 showed significantly better percentage lean, loin depth, and backfat depth in comparison to the Control Group. Further, Groups 1 and 2 also exhibited better loin depth than the Control Group.

TABLE 4

| | Control | | | |
|---|---|---|---|---|
| | Group | Group 1 | Group 2 | Group 3 |
| Percentage | 52.50 | 53.05 | 53.43 | 53.73 |
| Lean (%) | | | | |
| Loin Depth | 57.25 | 60.50 | 61.01 | 61.69 |
| (mm) | | | | |
| Backfat | 19.06 | 19.20 | 18.09 | 18.22 |
| Depth (mm) | | | | |

### EXAMPLE 2

In this example, the efficacy of a dietary acid composition in combination with a ractopamine hydrochloride product was examined.

Methods and Materials

A total of six tests were performed at six different farm locations. In each test, the pigs were divided into different treatment groups. Group I was fed a dietary acid composition, which, for this example, was commercially-available Opti-Pak Efficiency™. Group II was fed a ractopamine hydrochloride product which, for this example, was commercially-available PayLean®. Group III was fed a combination of both the dietary acid composition and the ractopamine hydrochloride product. The Control Group was feed a complete feed without either of the dietary acid composition or the ractopamine hydrochloride product. It should be noted that not all treatments groups are represented in each field test. The treatments examined in each field test differed depending on circumstances related to the farming operation.

In each case, various calculations were made for each group, such as average daily gain, feed:gain ratios, average daily feed intake, backfat, % lean, and % yield. The actual calculations varied from test to test, as shown below in the results.

Results

Table 5 shows the results of a test performed by a pork producer in Illinois involving 1,770 head of grower/finisher pigs. Please note that the ractopamine hydrochloride product fed to Group 2 was fed at 4.5 grams for the first 21 days of the test and at 9 grams for the subsequent 14 days.

TABLE 5

| | Group 1 | Group 2 | Group 3 |
|---|---|---|---|
| Gilts, ADG | No Data | 1.97 | 1.84 |
| Barrows, ADG | 1.81 | 1.77 | 1.80 |

Table 6 shows the results of a test performed by a pork producer in Minnesota involving 3,171 head of grower/finisher pigs. Please note that the ractopamine hydrochloride product fed to Group 2 was fed at 4.5 grams for 28 days.

TABLE 6

| | Group 1 | Group 2 | Group 3 |
|---|---|---|---|
| ADG | 1.74 | 1.82 | 1.70 |
| F/G | 2.90 | 2.74 | 2.83 |
| ADFI | 5.05 | 4.98 | 4.80 |
| Backfat, inches | .80 | .82 | .69 |
| % Lean | 53.3 | 53.3 | 54.9 |
| % Yield | 76.4 | 76.7 | 75.8 |

Table 7 shows the results of a test performed by a pork producer in Minnesota involving 4,137 head of grower/finisher pigs. Please note that the ractopamine hydrochloride product fed to Group 2 was fed at 4.5 grams for 28 days.

TABLE 7

| | Group 1 | Group 2 | Group 3 |
|---|---|---|---|
| ADG | 1.67 | 1.79 | 1.63 |
| F/G | 2.90 | 2.72 | 2.77 |
| ADFI | 4.87 | 4.86 | 4.53 |
| Backfat, inches | .76 | .76 | .68 |
| % Lean | 53.6 | 54.0 | 55.1 |
| % Yield | 76.3 | 76.4 | 76.8 |

Table 8 shows the results of a test performed by a pork producer in eastern Iowa involving 6,008 head of pigs from wean to finish.

TABLE 8

| | Control | Group 1 | Group 2 |
|---|---|---|---|
| ADG | 1.63 | 1.65 | 1.49 |
| F/G* | 2.49 | 2.47 (2.38)* | 2.37 |
| Wt Mkt. | 249 | 263 | 248 |
| Culls Sold | 19 | 22.5 | 50 |
| Death Loss % | 1.59 | 1.95 | 2.75 |

*Adjusted to ending weight on Opti-Pak Efficiency ™ if sold at 249 lbs.

Table 9 shows the results of a test performed by a pork producer in Iowa involving 2,341 head of grower/finisher pigs.

TABLE 9

| | Control | Group 1 |
|---|---|---|
| ADG | 1.70 | 1.77 |
| F/G | 3.21 | 3.13 |
| ADFI | 5.47 | 5.54 |
| Death Loss % | 4.81 | 2.47 |

Table 10 shows the results of a test performed by a pork producer in northwest Iowa involving 4,378 head of grower/finisher pigs.

TABLE 10

| | Group 1 | Group 2 |
|---|---|---|
| ADG | 1.67 | 1.52 |
| F/G | 3.03 | 3.12 |
| ADFI | 5.09 | 4.73 |
| Death Loss % | 4.29 | 4.81 |

What is claimed is:

1. An animal dietary supplement comprising:
(a) a dietary acid; and
(b) a diet restriction component.

2. The supplement of claim 1, wherein the dietary acid is configured such that a portion of the dietary acid is active in the large intestine of the animal.

3. The supplement of claim 1, wherein the supplement is a delayed action supplement.

4. The supplement of claim 1, further comprising a protection component.

5. The supplement of claim 4, wherein the protection component is associated with the dietary acid.

6. The supplement of claim 1, wherein the dietary acid is encapsulated.

7. The supplement of claim 1, wherein the dietary acid comprises fumaric acid.

8. The supplement of claim 1, wherein the dietary acid comprises at least one acid selected from the group consisting of fumaric acid, malic acid, citric acid, phosphoric acid, lactic acid, formic acid, acetic acid, propionic acid, butyric acid, sorbic acid, tartaric acid, benzoic acid, and 2-hydroxy-4-(methylthio)butanoic acid.

9. The supplement of claim 1, wherein the diet restriction component comprises a beta-adrenergic agonist.

10. The supplement of claim 9, wherein the beta-adrenergic agonist comprises ractopamine hydrochloride.

11. The supplement of claim 1, wherein the diet restriction component comprises potassium chloride.

12. The supplement of claim 1, wherein the diet restriction component comprises at least one diet restriction component selected from the group consisting of a beta-adrenergic agonist, potassium chloride, bitter feed flavors, aromatics, salt, fat, distiller's grains, meat meal, and fish oil.

13. An animal dietary supplement comprising:
(a) a dietary acid;
(b) a diet restriction component selected from the group consisting of potassium chloride, bitter feed flavors, aromatics, salt, fat, distiller's grains, meat meal, and fish oil; and
(c) a protection component.

14. A method of feeding an animal, the method comprising:
providing a dietary acid to an animal as part of a restricted diet.

15. The method of claim 13, wherein the animal is a swine.

16. The method of claim 14, wherein the swine is a growing/finishing pig.

17. The method of claim 14, wherein the swine is a lactating sow.

18. The method of claim 13, wherein the animal is a non-ruminant.

19. A method of improving average daily gain in swine, the method comprising:
providing a dietary acid to a finishing swine as part of a restricted diet.

20. The method of claim 18, wherein the swine is a finishing pig.

21. The method of claim 18, wherein the swine is a lactating sow.

22. A method of improving feed efficiency in swine, the method comprising:
implementing a limited diet for a finishing swine; and
supplementing the limited diet with a dietary acid.

23. The method of claim 21, wherein the swine is a finishing pig.

24. The method of claim 21, wherein the swine is a lactating sow.

25. A method of improving feed efficiency in swine, the method comprising:
providing a dietary acid to a finishing swine as part of a restricted diet.

26. The method of claim 24, wherein the swine is a finishing pig.

27. The method of claim 24, wherein the swine is a lactating sow.

28. The method of claim 24, wherein the dietary acid is encapsulated.

29. The method of claim 27, wherein the encapsulated dietary acid is not fully released until it reaches a large intestine of the swine.

30. A method of improving leanness in swine, the method comprising:
providing a dietary acid to a finishing swine as part of a restricted diet.

31. The method of claim 30, wherein the swine is a finishing pig.
